(19) United States
(12) Patent Application Publication
(10) Pub. No.: US 2005/0256398 A1
(43) Pub. Date: Nov. 17, 2005

(54) Systems and methods for interventional medicine

(76) Inventors: Roger N. Hastings, Maple Grove, MN (US); Rogers C. Riters, Charlottesville, VA (US); Peter R. Werp, St. Louis, MO (US); Andrew F. Hall, St. Charles, MO (US); Walter M. Blume, Webster Groves, MO (US); John Rauch, St. Louis, MO (US); Scott G. Klimek, Spring Lake Park, MO (US); Raju R. Viswanathan, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 10/844,056

(22) Filed: May 12, 2004

(51) Int. Cl. A61B 5/05

(52) U.S. Cl. 600/423; 600/424; 600/410

(57) ABSTRACT

An automated system for navigating a medical device through the lumens and cavities in an operating region in a patient. The system includes an elongate medical device, having a proximal end and a distal end adapted to be introduced into the operating region. The system also includes an imaging system for displaying an image of the operating region, including a representation of the distal end of the medical device in the operating region. The system also includes a localization system for determining the position of the medical device in a frame of reference translatable to the displayed image of the imaging system. Finally, the system includes a system for orienting the medical device in a selected direction in the operating region, this system may be, for example, a magnetic navigation system which acts through the interaction of magnetic fields associated with the medical device inside the operating region and at least one external source magnet outside the patient's body.

### CROSS REFERENCE TO RELATED APPLICATION

This application claims priority of prior PCT Application Serial No. PCT/US03/10893, filed Apr. 9, 2003, which claimed priority of Provisional U.S. Application Ser. No. 60/371,555, filed Apr. 10, 2002, the entire disclosures of which are incorporated herein by reference.

### FIELD OF THE INVENTION

This invention relates to a system and methods for interventional medicine.

### BACKGROUND OF THE INVENTION

Interventional medicine is the collection of medical procedures in which access to the site of treatment is made through one of the patient's blood vessels, body cavities or lumens. For example, angioplasty of a coronary artery is most often performed using a catheter which enters the patient's arterial system through a puncture of the femoral artery in the groin area. The procedure is referred to as PTCA, or Percutaneous (through the skin), Transluminal (through the blood vessel), Coronary (in the vessel of the heart), Angioplasty. Other interventional medical procedures include assessment and treatment of tissues on the inner surfaces of the heart (endocardial surfaces) accessed via peripheral veins or arteries, treatment of vascular defects such as cerebral aneurysms, removal of embolic clots and debris from vessels, treatment of tumors via vascular access, endoscopy of the intestinal tract, etc .

Interventional medicine technologies have been applied to manipulation of instruments which contact tissues during surgical procedures, making these procedures more precise, repeatable and less dependent of the device manipulation skills of the physician. However, currently available interventional medicine systems do not provide adequate manipulation of the distal tip of a medical device for conducting many diagnostic and therapeutic procedures.

Many presently available interventional medical systems for directing and manipulating the distal tip of the catheter device from the proximal end of catheter use pull wires to deflect the distal tip, and rely upon torque transmitted from the proximal end to rotate the deflected tip to the desired orientation. If the device has made significant bends along the path from the entrance site to the treatment site, torque may not be transmitted in a predictable manner from the physician's hands to the device tip. The pull wires tend to make the distal end of the device stiff, and they occupy valuable space in the small cross sectional area of the device, which must often contain other devices for diagnosis or treatment. The lack of a predictable response of the catheter distal tip to movements at the proximal end, prevents the use of deterministic equations to guide the device.

### SUMMARY OF THE INVENTION

According to the principles of the present invention, a preferred approach involves the direct manipulation of the distal tip via coupling from an external energy source. One approach uses electrical energy transmitted through fine wires embedded in the catheter walls to activate piezoelectrics or electrostrictive polymers, such as disclosed in Cheng et al, App. Phys. Lett. Vol 74, pp. 1901-1903 (1999), incorporated herein by reference. Another approach, which completely avoids mechanical or electrical links through the length of the catheter, uses magnetic coupling between a small magnet in the catheter tip, and large magnets external to the patient that generate computer controlled magnetic fields at the catheter tip, see for example U.S. Pat. No. 4,869,247, “Video Tumor Fighting System”, U.S. Pat. No. 5,125,888, “Magnetic Stereotactic System for Treatment Delivery”; and U.S. patent application Ser. No. 09/405,314, “Cardiac Methods and System”), the disclosures of each of which are incorporated herein by reference. A variation on the use of magnetic coupling uses a fixed external magnetic field coupled to a set of coils in the tip of the device capable of generating a variable magnetic moment for actuation and steering the tip, as disclosed in U.S. patent application Ser. No. 09/504,835 “Magnetic Medical Devices with Changeable Magnetic Moments and Methods of Navigating Magnetic Medical Devices with Changeable Magnetic Moments”; U.S. patent application Ser. No. 09/772,188 “Catheter Navigation Within an MRI Imaging Device”; U.S. Pat. No. 6,304,769 “Magnetically Directable Remote Guidance Systems, and Methods of Use Thereof”; and U.S. Pat. No. 6,216,026, “Method of Navigating a Magnetic Object, and MR Device”, Kuhn et al.), the disclosures of each of which are incorporated herein by reference.

The present system and method provide clear and unambiguous images of the tissues in the operating region, including an image or accurate representation of the catheter device. The present system and method can also provide the capability for operation from a remote location, removing the physician from the procedure site and thereby reducing exposure to X-rays or high magnetic fields from imaging devices. Remote operation also allows a particularly skilled physicians to operate over a broader geographical area.

### BRIEF DESCRIPTION OF THE DRAWINGS

FIG. 1 is a schematic diagram of an automated system for navigating a medical device through the lumens and cavities in the operating regions in a patient in accordance with the principles of this invention;

FIG. 2 is a block diagram of the system;

FIG. 3 is a schematic diagram of a magnet and magnetic medical device;

FIG. 4 is a perspective view of a magnet system for creating a magnetic field in a patient for magnetic navigation;

FIG. 5A is a top plan view of the distal end of a magnetic medical device constructed according to the principles of this invention, employing employing coils in the distal end of the medical device for magnetic navigation;

FIG. 5B is a side elevation view of the distal end of the magnetic medical device shown in FIG. 5A;

FIG. 5C is a transverse cross-sectional view of the distal end of the magnetic medical device, taken along the line of 5C-5C in FIG. 5B;

FIG. 6A is a side elevation view of the distal end portion of a medical device incorporating an electrostrictive element;

FIG. 6B is a transverse cross-sectional view of the distal end portion of a medical device, taken along the plane of line 6A-6A;

FIG. 6C is a schematic side elevation view of the distal end portion of a medical device, showing one electrostrictive element thereon for changing the shape of the device upon the application of electric potential;

FIG. 6D is a schematic side elevation view of the distal end portion of a medical device, showing multiple electrostrictive elements thereon for changing the shape of the device upon the application of electric potential;

FIG. 6E is a schematic side elevation view of the distal end portion of a medical device, showing a compound electrostrictive element thereon for changing the shape of the device upon the application of electric potential, to the first, the second, or to both parts of the compound electrostrictive element;

FIG. 6F is a schematic side elevation view of the distal end portion of a medical device, showing two longitudinally extending compound electrostrictive elements for changing the shape of the device upon the application of electric potential;

FIG. 6G is a schematic side elevation view of the distal end portion of a medical device, showing a mosaic electrostrictive elements for changing the shape of the device upon the application of electric potential;

FIG. 7A is a side elevation view of the distal end portion of a medical device incorporating a magnetostrictive element;

FIG. 7B is a transverse cross-sectional view of the distal end portion of a medical device taken along the plane of line 7A-7A.

FIG. 8A is a perspective view of an advancer unit, as it would be mounted on a support bracket; and

FIG. 8B is a perspective view of an advancer system, including the control, drive, and advancer unit;

FIG. 9 shows a schematic representation of the device geometry; and

FIG. 10 is a process flow chart for the feedback controlled navigation system.

### DETAILED DESCRIPTION OF THE INVENTION

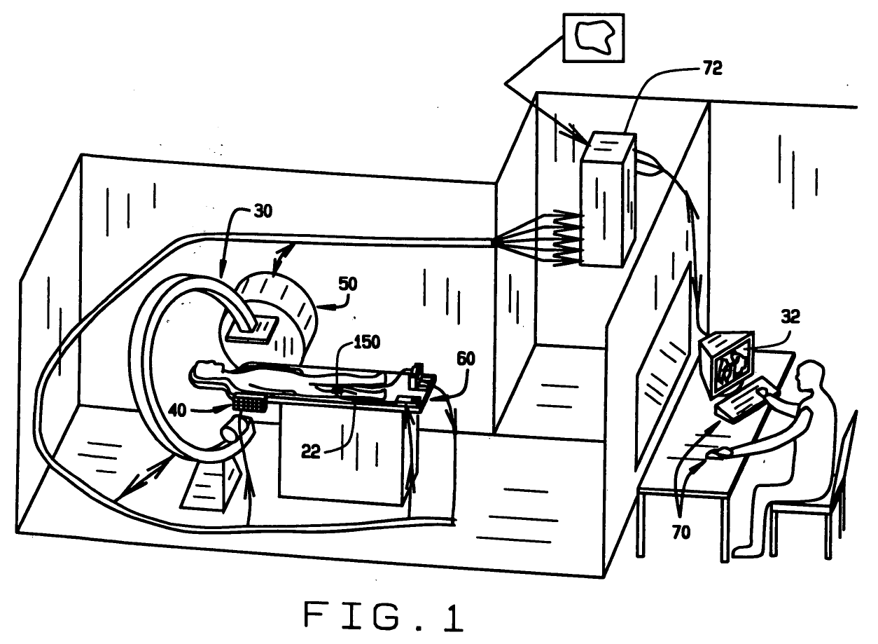

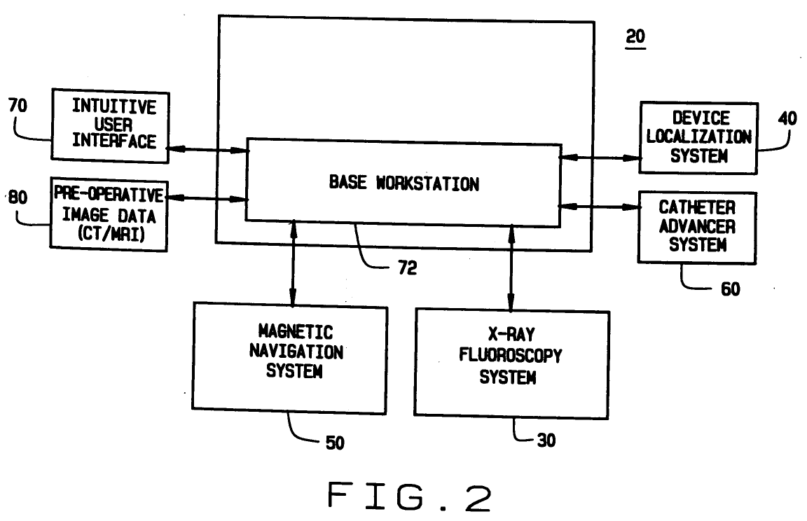

An automated system for navigating a medical device through the lumens and cavities in an operating region in a patient in accordance with the principles of this invention is indicated generally as 20 in FIG. 1. The system 20 comprises an elongate medical device 22, having a proximal end 24 and a distal end 26 adapted to be introduced into the operating region O in a patient P. The system 20 also comprises an imaging system 30 for displaying an image of the operating region O on a display 32, including a representation of the distal end 26 of the medical device 22 in the operating region O.

The system 20 also includes a localization system 40 for determining the position of the medical device 22 in a frame of reference translatable to the displayed image of the imaging system.

The system 20 also includes a navigation system for manipulating the distal end 26 of the medical device 22. In this preferred embodiment the navigating system is a magnetic navigation system 50. Of course, as discussed above, the navigation system could alternatively be a piezoelectric system or a mechanical system. The magnetic navigation system 50 orients the distal end 26 of the medical device 22 in a selected direction in the operating region O through the interaction of magnetic fields associated with the medical device 22 inside the operating region O and at least one external source magnet outside the patient's body.

The system 20 also comprises an advancer 60 acting on the medical device adjacent the proximal end 24 of the medical device 22 for selectively advancing the distal end 26 of the medical device 22.

Finally, the system comprise an input device 70 for receiving at least a destination for the distal end 26 of the device 22 input by the user using the displayed image from the imaging system 30. A controller 72, responsive to the destination input by the user, operates the magnetic navigation system 50 to orient the distal end 26 of the medical device 22 in the proper orientation to reach the input destination, and as the distal end 26 of the medical device 22 moves to is in the proper orientation, operating the advancer 60 to advance the distal end 26 of the medical device 22 to the destination input by the user.

The imaging system 30 may be an x-ray, MRI, ultrasound, or other imaging system. Most interventional procedures currently use fluoroscopy for imaging of the blood vessels, tissue surfaces, and catheter devices. Dye or contrast agents which absorb X-rays are injected through the catheter system to obtain a “roadmap” of the vessels or outline of the endocardial surfaces. The medical devices 22 can contain radiopaque materials such as platinum marker bands or polymers loaded with heavy metals to render them visible in the fluoroscopic image. The physician views the tissues and the medical device in a single imaging plane, which does not uniquely specify the orientation of the catheter or the direction of tissue targets. The single imaging plane must be rotated to two or more angles to generate a perspective view. Many imaging systems contain two planes of imaging, often configured for orthogonal views. Some modern fluoroscopic systems have the capability to capture images while rotating around the patient. Three dimensional images are then quickly reconstructed by the imaging system. While these images are not quite “real time” because of the time required for rotation and computing, the three dimensional images are most useful for catheter navigation.

Magnetic Resonance Imaging (MRI) measures the density of protons in the tissue and catheter device, and generates a three dimensional image. Traditionally, MRI images have required considerable time for computer processing, and have thus been used for tissue diagnostics. Improvements in computing rates are now allowing the “real time” use of MRI to guide catheter devices. The magnetic medical devices must be non-magnetic during imaging to avoid blurring, or have a slightly magnetic property to make them visible in the image.

Ultrasound imaging has been used for real time imaging from outside the patient. Image resolution is generally inferior to fluoroscopic and MRI images.

Pre-operative images can be used in combination with accurate device localization, provided that the tissue being navigated does not move or change during the procedure. Pre-operative images from a pre-operative imaging system 80, such as a high resolution MRI or CT scan, can be used in combination with real-time imaging to provide improved anatomical roadmaps. Moving real time tissue images, for example heart tissue, can be enhanced by dynamic “registration” of the pre-operative image to the real time image. The registration process compares the real-time two dimensional image to 2-D projections of the three dimensional pre-operative image, and selects the best match to achieve registration. Tissue which does not have salient features, for example a roughly spherical, enlarged heart, can be difficult to uniquely match in this way. However, adjoining features, such as the pulmonary vein structure entering the left atrium of the heart, may have an unambiguous three dimensional structure, and can be included in the registration process to ensure a unique match of pre-operative and real-time images.

Local imaging can be used alone or in combination with global imaging. Interventional and intravascular ultrasound catheters have been developed for diagnostics and catheter guidance applications. Endoscopes are commonly used in interventional procedures. Regardless of the imaging modality, a digital or digitized image is required in an interventional robotics system. The pixel or voxel resolution will contribute to the overall accuracy of the robotic guidance system.

The device localization system 40 can be magnetic, electric, or ultrasonic. The computer must be fed accurate information on the location of the catheter device relative to the tissue. Many technologies have been developed for this purpose. For example, image processing allows the catheter tip to be located to within pixel accuracy on the two images in a bi-plane fluoroscopic system. Similarly, the three dimensional MRI image contains the location of the catheter relative to tissue, again with voxel accuracy. Localization sensors or transmitters may be placed in the catheter tip to provide localization relative to set of external transmitters or receivers, allowing a triangulation of catheter tip location. Such systems often employ low frequency electromagnetic signals that penetrate body tissues without distortion, or signals generated by ultrasound transceivers.

As described above, the navigation system can comprise an electrically or magnetically active device for articulating the distal end 26 of the medical device 22. Articulation can refer to a variety of movements of the medical device 22 along its length, changes is shape or configuration, actuation of therapeutic mechanisms at the distal tip 26, or even to untethered devices that “swim” through the body under remote control. In this preferred embodiment the device 22 is an elongate medical device with a distal tip 26 that is articulated for steering and navigation of the device 22, however those skilled in the art will recognize that the systems and methods of the present invention could be applied more broadly.

As shown in FIG. 3, in one preferred method of articulating the distal tip 26, a small magnet 52 is disposed within the distal tip, with large controlling magnets 54 placed outside the patient. The orientation of the magnetic field at the site of the distal tip 26 is computer-controlled by adjusting the orientation of the external permanent magnets 54 or adjusting the currents in external electromagnets. The tip magnet 52 aligns with the direction of the externally applied magnetic field direction, thus allowing the catheter tip 26 to be steered to any direction in three dimensional space. Prior art, manually articulated catheters, can typically be deflected in only one plane, so that steering to a given point in space involves first deflecting the catheter tip to the side, then twisting the catheter to rotate the tip to the desired point. This cumbersome navigation means makes precise navigation of the tip difficult or impossible. In the present invention, the catheter tip is deflected in a plane that contains the target site, so that the tip moves smoothly and precisely to the target. With this mode, there are no wires or connections to the tip magnet, and the cross-sectional area of the medical device 22 is available along the entire length of the device proximal to the tip magnet 52 for therapeutic or diagnostic means. The magnet 52 can contain holes or slots to accommodate wires or movement of fluids to the distal catheter tip. Tip magnet steering allows the portion of the medical device to proximal to the magnet 52 to be free of stiffening wires and mechanisms, enabling a highly flexible segment proximal to the magnet. When the catheter tip is pushed into tissue, this flexible segment will buckle, thus reducing the risk of tissue perforation by the tip of the medical device.

One embodiment of a combination of an imaging system 30 and a magnetic navigation system 50 is shown in FIG. 4 as it would be arranged in a procedure room to perform an interventional medical procedure on a patent P supported on a support 90. As shown in FIG. 3, the imaging system 30 comprises a conventional C-arm 92 that allows rotation of the imaging equipment about three mutually perpendicular axes. An x-ray source 94 and an imaging plate 96 are mounted on the C-arm 92, with the x-ray source 94 opposite the imaging plate 96 for imaging the portion of a patient's body between the source and the imaging plate. The magnet system 30 preferably comprises two rotating and pivoting magnets 98 and 100 mounted on opposite sides of the patient for creating a magnetic field of variable direction inside the patient to navigate a medical device 22 therein by projecting a field in a selected direction that the distal end of the medical device 22 aligns with.

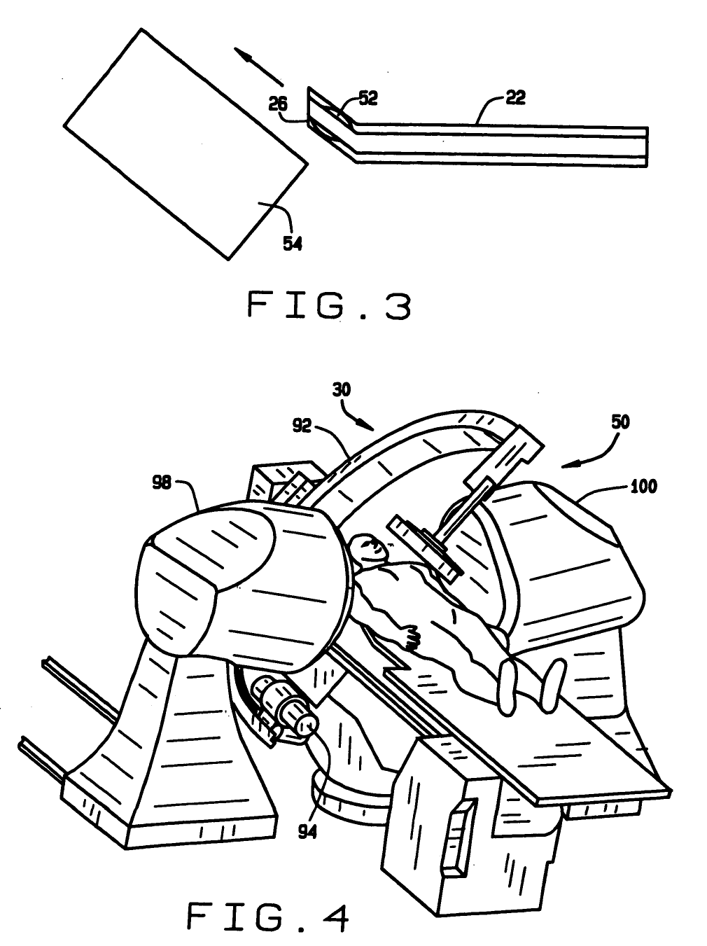

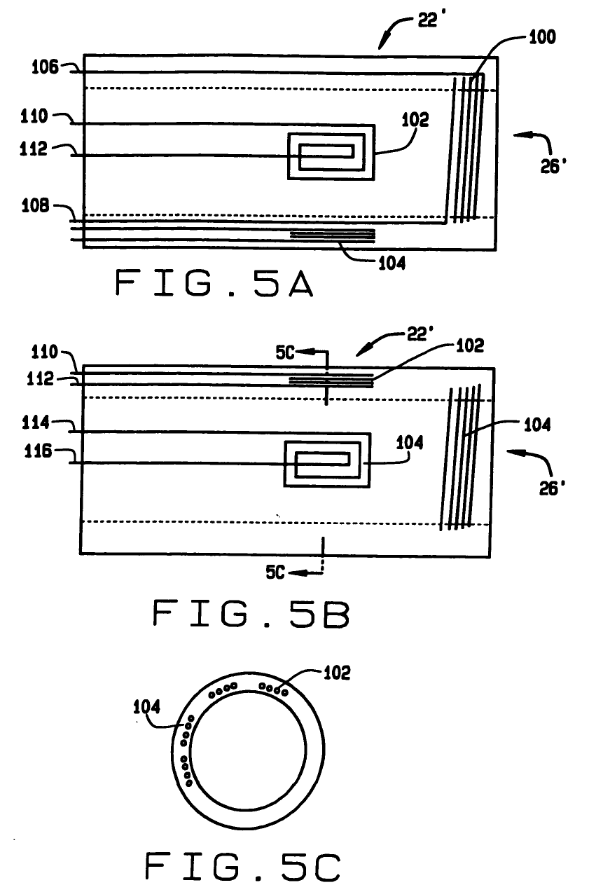

Another means for navigating the distal tip of a medical device uses a fixed external magnetic field, and a variable direction magnetic moment in the catheter tip. While a variety of means exist to create a variable tip moment, a preferred means uses a set of three mutually orthogonal electrical coils in the catheter tip, which can generate a moment in any direction in space. As shown in FIGS. 5A, 5B and 5C an alternative construction of the medical device, indicated as 22′. The distal end 26′ of the medical device 22′ is provided with a set of three coils 100, 102, and 104, which are preferably arranged so that axes of the coils are mutually perpendicular. As shown in FIGS. 5A, 5B, and 5C, the turns of coil 100 extend circumferentially around the distal end 26′ of the device 22′, and are preferably embedded in the sidewall thereof. The turns of coil 102 are arranged so that axis of the coil is oriented in a radial direction, and are preferably embedded in the sidewall as well. Similarly, the turns of coil 104 are arranged so that the axis of the coil is oriented in a radial direction (preferably rotated 90° circumferentially with respect to coil 102), and are preferably embedded in the sidewall as well. Leads 106 and 108 extend to coil 100, leads 110 and 112 extend to coil 102, and leads 114 and 116 extend to coil 104 to selectively apply power to the coils to create local magnetic moments. The moment created by the coils 100, 102, and 104 in the distal end 26′ of the medical device 22′ aligns with the external field applied by external magnets to deflect the distal tip 26′ of the device 22′. An example of such a device is that disclosed in U.S. patent application Ser. No. 09/504,836, filed Feb. 16, 2000, incorporated herein by reference.

The external magnet can be any strong magnet source, for example as disclosed in Kuhn, U.S. Pat. No. 6,216,026, and Arensen, U.S. Pat. No. 6,304,769, the disclosures of which are incorporated herein by reference. When variable moment navigation is used with the fixed field of an MRI imaging device, the moment currents in the medical device are zeroed between navigation steps, rendering the catheter tip non-magnetic for MRI imaging of the adjacent tissue. Another special consideration in the fixed external field mode is the inability to apply torque about the direction of the fixed field, since the torque is always orthogonal to the plane of the field and moment vectors. Special serpentine paths need to be navigated to torque the catheter about the fixed field direction, as disclosed in U.S. patent application Ser. No. 09/772,188, filed Jan. 29, 2001, and incorporated herein by reference. Alternatively, a second fixed field direction can be used for this purpose. This navigation problem does not exist in the variable field mode, since torque is generally not required about the fixed moment direction (along the catheter length).

Another means for navigating the distal tip of a medical device uses electrostrictive polymers to deflect the catheter tip, also requires electrical leads to extend from the proximal end to distal end of the catheter. Electrostrictive materials are those in which an applied magnetic field will result in a strain of some kind (expansion, contraction or twisting). These same materials usually behave also as piezoelectric devices, which when compressed, stretched or twisted will create electric fields which constitute a voltage between certain points on the material's surface. Commercial elements are available, made from these materials. Here we discuss the more specific electrical and mechanical arrangements of material for their use in navigation procedures with medical devices.

Electrostrictive behavior is a means of controlling the distal region of a medical device (e.g. catheter, endoscope, guidewire or electrode) for navigation in the body. (Herein we use “catheter” to represent any such medical device.) This requires the application across some segment of the material of a controlling voltage or voltages from external source(s) to create the desired strains and consequent bending of the catheter. Little energy is required in electrostrictive activity (current only flows while the molecular constituents achieve their strained condition or while changing the strained condition). Fine wires can be used, but one skilled in electromagnetism would know how to insulate them so that high voltages would not cause arcing or otherwise endanger a patient.

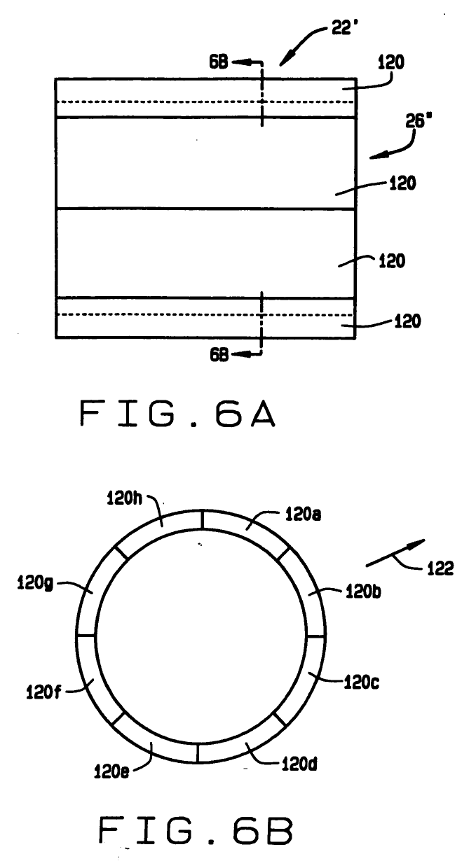

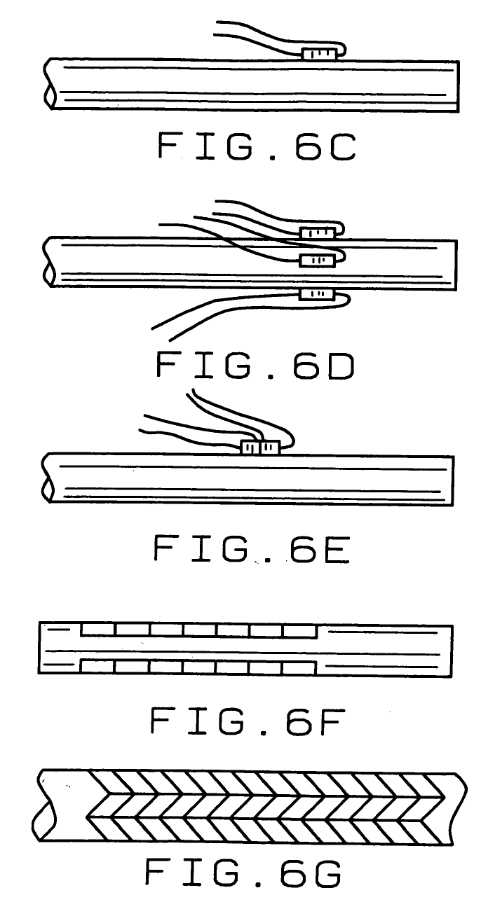

An alternative embodiment of the medical device incorporating an electrostrictive element is indicated generally as 22″ in FIGS. 6A and 6B. As shown in FIGS. 6A and 6B, the distal end 26″ of the medical device 22″ is provided with a plurality of electrostrictive elements 120. In this preferred embodiment, there are eight electrostrive elements 120a-h, spaced around the circumference of the medical device 22″, and oriented parallel with the longitudinal axis of the of the medical device. The application of an electric potential across the electrostrive element causes the electrostrictive element to change dimension, causing the medical device to bend. By selectively activating one or more of the electrostrictive element 120, the distal end 26″ of the medical device 22″ can be bent in a selected direction. Thus, for example to move bend the distal end 26″ of the device 22″ in direction of arrow 122, an electric potential is applied to element 120b, or to elements 120a, 120b, and 120c, to cause the distal end to bend. An advantage of the use of electrostrictive elements is the that external magnetic fields are not required.

A simple means of employing electrostriction might be a single element operating a joint or flexible spot near the distal end of a catheter. This could be cemented to the external wall of the catheter as shown in FIG. 6C below. The bending action at the wall could occur either because the device itself would bend upon application of voltage, or by cementing an element to another element which could bend but not stretch or compress. Application of a voltage to the two fine wires (small current required) would activate the element which would bend the catheter. This could then be twisted at the proximal end to effect a turn in any direction. Such a device would be limited in its capabilities, however.

A more preferred device might consist of multiple (2, 3, 4, or more) elements cemented about the catheter as indicated in FIG. 6D. Combinations of voltages could then turn the catheter in any direction without being twisted at the proximal end. Since elements can constrict or expand depending on the voltage polarity, two orthogonal elements would be sufficient to turn in any direction, but such a limited array might not be efficient. For example, such an arrangement would result in asymmetry at that point of the catheter, which might cause uneven effectiveness in some turning directions, requiring additional effort to executed the desired turn.

A still more preferred device might consist of multiple elements acting mechanically in series but electrically in parallel for each turning element as shown in FIG. 6E for a single turning component having two elements in this fashion. This would increase the available bend angle without requiring greater voltage. The wires shown here to effect this type of electrical connection, might be built into an insulating catheter wall.

A useful form of a turning element for a given direction, might be extended series of elements, many more than the two of FIG. 6E. These would act not at a short turning directional location along a catheter as in FIGS. 6C, 6D, or 6E, but instead apply a bending turn over a significant distance along a side of a catheter. In this case, each element of a strip would act mechanically in series with a next element of the same strip, but electrically in parallel, an extension of the simpler method of FIG. 6E. Possibly 3 or 4 such strips, as in FIG. 6F, would be more effective in turning a catheter, allowing a longer arc if desired. The construction of such strips might take advantage of methods used in the semiconductor industry. A modification of this embodiment might consist of having a variable transfer function (ratio of bend to applied voltage) along each strip so as to provide for a non-uniform bend of a catheter of uniform bending stiffness. That is, each elemental one of the mechanical series would have a different strain from its neighbor. Of course, the same effect could be made with a catheter of variable stiffness and uniform electrostrictive strips, but that might be undesirable for other medical reasons in some applications.

Another embodiment of a medical device incorporating electrostrictive control element is shown in FIG. 6G, in which a mosaic array of electrostrictive elements is distributed along and around a catheter. The interconnections (using, for example methods of the semiconductor industry) could be designed to optimize different catheters for different functions. Insulating gaps between elements would allow compression or expansion. Alternatively, the elements might be tiny wires might be buried in insulating catheter walls. Alternatively, the mosaic might be general in distribution of elements, but computer control of multiple feed-wires. could provide considerable functional variability. The catheter and its control could act in a “smart” fashion to optimize a given catheter design for different requirements in a given type of procedure.

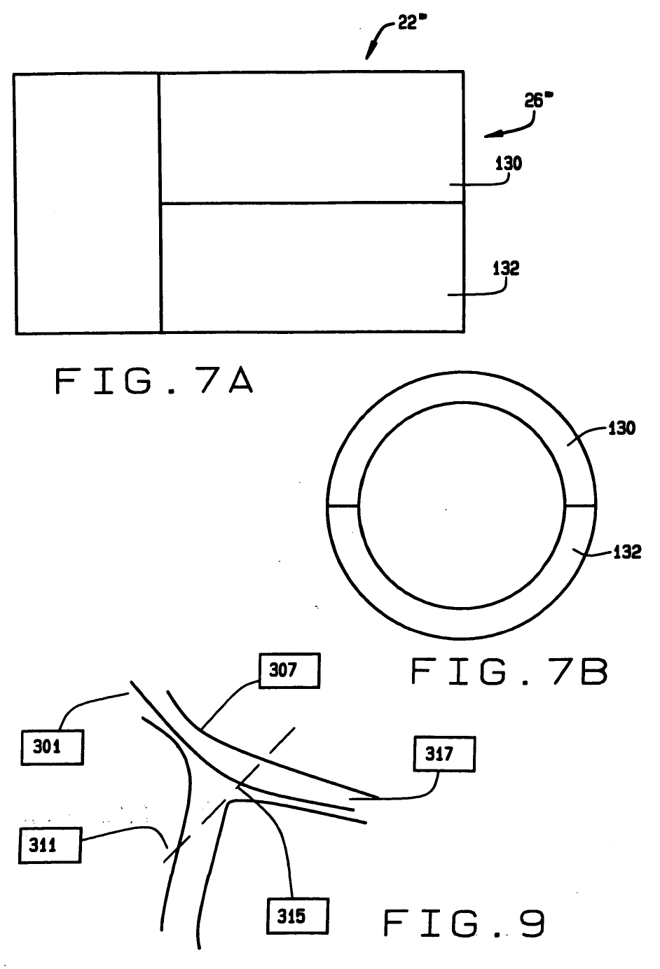

Still another means for navigating the distal tip of a medical device uses magnetostrictive materials. Local magnetic fields generated by coils near the catheter tip (or external fields) could be used to deflect a magnetostrictive material in the catheter tip. Similarly, a deflection joint can be constructed from a permanent magnet, for example using a spherical magnet ball in a plastic socket, which is deflected using magnetic fields generated either by coils within the catheter or by external magnetic fields, similar to the devices and methods disclosed in U.S. patent application Ser. No. U.S. patent application Ser. No. 09/504,836, filed Feb. 16, 2000, for Magnetic Navigation of Medical Devices in Applied Magnetic Fields, the disclosure of which is incorporated herein by reference. An alternative embodiment of the medical device incorporating an magnetostrictive element is indicated generally as 22′″ in FIGS. 7A and 7B.

As shown in FIGS. 7A and 7B, distal end 26′″ of the medical device 22′″ comprises a first section 130 of a magnetostrictive material, and a second section 132 of a non-magnetostrictive material. In this embodiment the first and second sections 130 and 132 are semi-cylindrical are joined together along common axially extending joints. The magnetostrictive material comprising the first section 130 elongates in the presence of a magnetic field, and the non-magnetostrictive material comprising the second section 124 does not. Thus, upon application of a magnetic field to the distal end 26′″ of the medical device 22′″, the distal end tends to bend away from the elongated first section 130. By a combination of the application of a magnetic field and axial rotation of the distal end of the device 22′″, the end of the device can be turned within a large range of directions.

In some cases, combinations of the above deflection methods might be useful. For example, a single axis of electrostrictive torque could be activated when torque cannot be provided about the field axis in an MRI imaging device.

The imaging system 30, localization system 40, and navigation system 50 of the system 20 are defined relative to one or more spatial coordinate systems, which must be “registered” to one another within the controller 72. Fluoroscopic or MRI images form the imaging system are viewed relative to a “tissue” or body coordinate system. When these images are used for device localization, then the device itself is registered properly with respect to the image. When the device is localized relative to the coordinate system of a set of transmitters, then this coordinate system must be registered to the image coordinate system to properly fuse the localization data to the image data. When pre-operative images are used, the device localization coordinate system must be registered to the pre-operative image. Pre-operative and real time images must be registered. If the image is moving, the registration must be dynamic, occurring at one or many time points. For example, an ECG signal can be used to register images at given points in the cardiac contraction cycle. Motion sensors on the chest can be used to register the images at given points in the respiration cycle. When external fields are used to steer the catheter, the field coordinates must be registered to the image and localization coordinates. Similarly, local images provided by ultrasound or endoscopes must be registered to field coordinates.

Registration can be done in part as a calibration process between fixed coordinate systems, for example field generation coordinates and localization transmitter coordinates. Anatomical landmarks or markers fixed to the patient can be used to register images, and to serve as references for localization. It may be necessary for the physician to facilitate registration, for example by pointing to anatomical landmarks on the patient with a localization wand. When the patient cannot be adequately immobilized, patient localization means may be required to account for his movements during the procedure, and automatically adjust the coordinate registration.

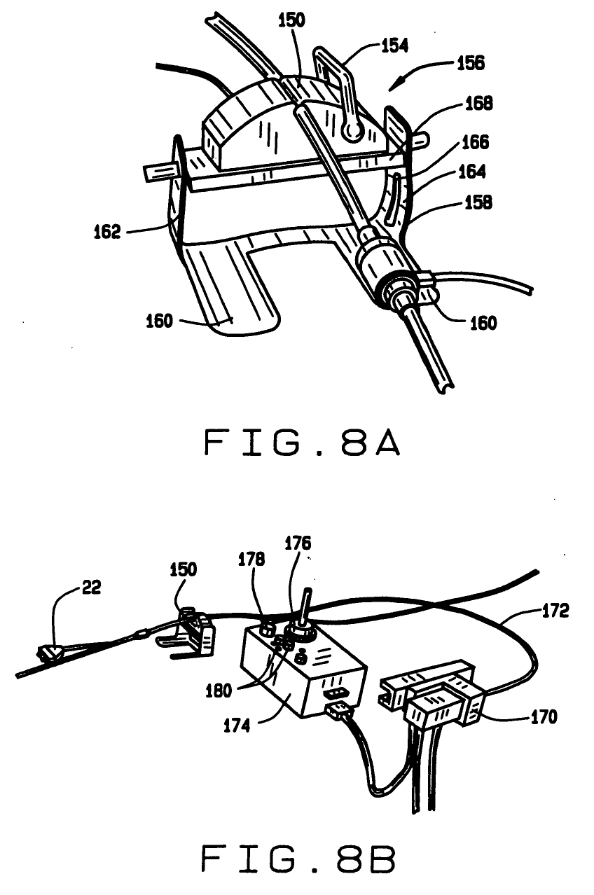

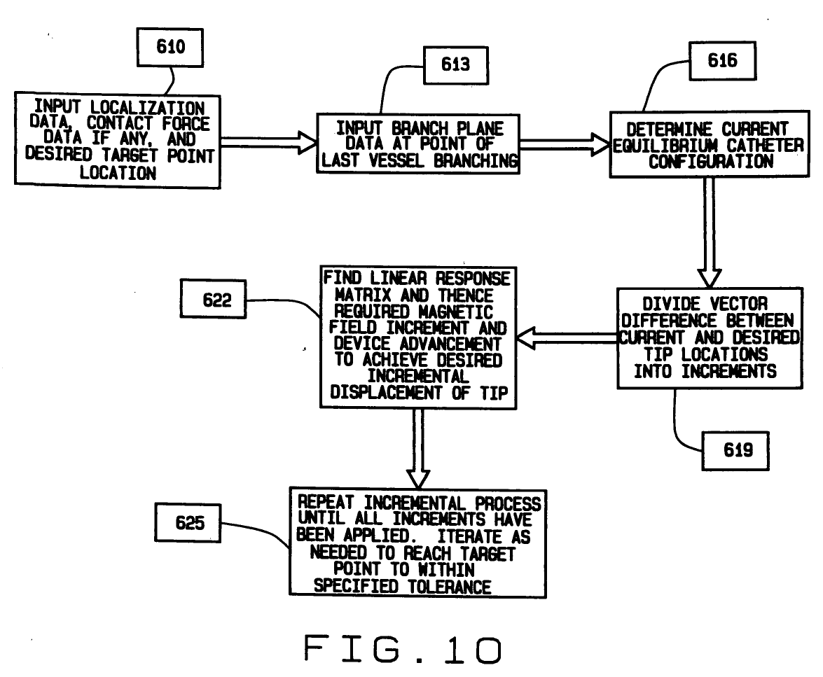

The advancer 60 may be similar to that disclosed in U.S. patent application Ser. No. 60/288,879, filed May 6, 2001, incorporated herein by reference, or some other suitable device, or as shown in FIGS. 8A and 8B. The advancer 60 preferably acts upon the proximal end 24 portion of the medical device 22 to advance the distal end 26 of the device in the direction in which it has been oriented. The advancer 60 is preferably controlled to only advance the distal tip after the distal tip as been located in appropriate direction, however coordinated actuation of deflection and advancement is possible. The advancer can be controller to begin movement as soon as the orientation of the medical device begins, or when the orientation of the device is within a specified error limit of the desired direction.

As shown in FIG. 8A the advancer 60 includes an advancer unit 150 having a slot 152 therein for receiving and drivingly engaging the proximal end portion of the medical device 22. A lever 154 operates the advancer unit 150 to open the slot 152 for inserting and removing the medical device 22. The advancer unit 150 is mounted on a support 156, comprising a bracket 158, having two tabs 160 for securing the support on or near the patient, and two upright members 162 and 164, each having a slot 166 therein. A tray 168 is mounted between the upright members 162 and 164 for pivotal and sliding movement in the slot so that the height and angular orientation of the tray, and thus the advancer unit 150 can be adjusted.

As shown in FIG. 8B the advancer 60 also includes a drive motor 170 connected to the advancer unit 150 via a flexible, sheathed drive cable 172. The drive motor 170, through the drive cable 172, drives wheels engaging the medical device in the advancer unit 150 to advance and retract the medical device. A control unit 174 is connected to the drive motor for controlling the operation of the drive motor. Of course, in addition to the control unit 174, or instead of the control unit 174, the drive motor could be connected to controller 72. The control unit 174 (and/or the control 72) can provide signals that cause the advancer unit to advance and retract the medical device 22, to control the speed of the advancement or retraction, or to control the advancement and retraction in specified increments for specified time periods or distances. The control unit 174 can have a joystick 176 for controlling the direction and speed of advancement. The control unit 174 can also have an emergency stop button 178, and buttons 180 for operating the motor to advance in predetermined increments. Where the control unit 72 is connected with the drive motor 170, the advance can be controlled under feed back from the imaging system and/or the localization system to automatically advance the distal end of the medical device to particular locations.

In conventional procedures, the physician manually advances the catheter, however it is preferable to replace the physician's hands with a computer controlled mechanical advancer. This allows the physician to operate from a radiation-safe environment, such as a remote control room. More importantly, it allows complete automation of the navigation of the medical device, which is the goal of an interventional robotics system. Coordination of steering and advancing moves can be complex, and is well-suited for computer control, giving the physician as much time and freedom as possible to concentrate on physiologic and therapeutic aspects of the procedure. The advancer 60 must provide safe and effective forward and reverse movements of the medical device which are guided by the localization system 40 and imaging system 30, and which coordinate with the steering system to automatically converge on physician-specified targets or to move in response to physician commands.

The physician interface preferably includes an input device 70, that allows the physician to input at least a desired destination. This input means preferably allows the physician to input the desired destination using the display 32 of the imaging system 30. The physician must control the interventional robotic system using a safe and efficient user interface. In the “telemetric” mode, the physician uses hand controls such as a joy stick to move the catheter while observing a real time image of the catheter and tissue. Voice activated controls are possible, as are visually activated controls which are actuated by the physician eye movements. In the “automatic” mode, the physician specifies an end-point or process, and the computer automatically causes the medical device to move. For example, the physician may point and click on an anatomical point on a three dimensional in the tissue image, and the computer would then advance and steer the catheter to the specified point.

Or the physician may define a path by touching points or drawing a line on the tissue image. Similarly, the physician may define a grid of points that the physician wants the catheter tip to touch in succession. In every case, the physician interface allows the doctor to interact with the tissue image to specify catheter movements, and to observe the subsequent movements of the catheter.

The control 72 preferably controls the navigation system to orient the distal tip 26 of the medical device 22 in the appropriate direction to reach the destination. The control 72 also preferably operates the advancer 60 while once the navigation system 50 orients the distal tip 26 in the appropriate direction, to advance the distal tip 26 of the medical device 22 to the destination selected by the physician. The control 72 includes a computer to manipulate the digital images; add catheter localization information to the images; interpret physician commands and translate these to catheter deflector commands; translate information between all relevant coordinate reference frames while coordinating auxiliary data such as ECG signals used to gate the images; control the advancer consistent with physician commands and coordinated with steering commands; supply data to the user interface monitors, and receive commands from the user interface. In the “automatic” mode, the computer must drive the catheter to a physician specified end point. This is accomplished with a closed loop algorithm that uses catheter localization data to successively reduce the distance between the catheter tip and the specified location.

The interventional robotics system of this invention allows the physician to automatically direct the tip of a catheter to points, or along a path, within body lumens or cavities of a patient. The physician interacts with a user interface that sends his commands to a control computer, and presents the physician with images of tissue in the operating region, including an image of the catheter. The control computer integrates and registers real time and pre-operative images, local images, and catheter location data, and commands and coordinates the actions of a catheter advancer and catheter tip steering mechanism. The physician can operate remotely from the patient to reduce his exposure to radiation or magnetic fields. Exquisite catheter manipulation skills are not required, and the physician can concentrate his attention on navigation commands and the delivery of therapy.

The method for using the system will depend upon the field of application. For example, in a typical intravascular navigation procedure a puncture is made and a sheath is introduced into an the femoral artery or vein in the groin area of the patient. A guiding catheter is introduced and placed into the ostium (opening) of the target artery. In some cases the guiding catheter is introduced over an introducer wire. In a preferred embodiment, the guiding catheter is attached to a Catheter Advancer, then advanced and steered by the system 20, with no need for an introducer wire. In an interventional cardiology application, the guiding catheter is steered to, and seated into, the ostium of a coronary artery. Because of differences in patient anatomy, numerous guiding catheter shapes must be kept in stock in the catheterization laboratory. Quite often, one or more catheter exchanges must be performed to find a shape that accommodates entry into the ostium. In the present invention, only one steerable catheter is required, and the system 20 advances the guide catheter tip into coronary ostium. Furthermore, in the prior art, the guiding catheter can pop out of the ostium, either due to the beating motions of the heart, or due to forces exerted on the guide catheter by devices which are advanced there through. In the present invention, the physician can command the System to automatically maintain the position of the guiding catheter tip relative to the ostium. As the heart beats, the advancer and steering mechanisms continuously adjust the catheter to maintain catheter tip position. Only the force required to maintain tip position is exerted by the System, avoiding the excessive forces that can be exerted by a physician pushing on the proximal end of the catheter to hold or regain catheter position in the ostium.

With the guiding catheter in place, a guide wire is typically introduced through the guiding catheter and into the coronary artery to the site of treatment. In the present invention, the guide wire may be attached to a second catheter advancer, and guided by the System to the target site. In some cases, it may be possible for the system 20 to steer the guide wire into the coronary artery without the need for a guiding catheter. Next, a working catheter such as a balloon catheter, a stent delivery system, or an atherectomy device is introduced over the guide wire. The working catheter may be attached to a third catheter advancer. In some cases the working catheter may be advanced by the system 20 without the need for a guide wire. The physician often coordinates movements of the wire and catheter to facilitate catheter advancement. The system 20 can be commanded by the physician to independently manipulate the wire and catheter.

While most arterial plaques are eccentric, lying entirely or mostly on one side of the arterial wall, in today's practice the entire wall is treated or at least affected by all devices. For example, balloons and stents treat the entire circumference of the arterial wall. In the present invention, the tip of the catheter can be steered selectively to the plaque, and treatment, for example plaque removal, can be applied, leaving the healthy portions of the vessel untreated and undamaged. Drugs can be delivered to the treatment site locally and selectively, for example to inhibit restenosis.

A As a second example of an application of system 20, is the use of the system in the introduction of catheters into the chambers of the heart to map electrical signals and deliver therapies such as tissue ablation or drug delivery. Again, the system 20 advances the catheter to specified points on the endocardial surface. In one example, the physician specifies a region of tissue that he would like to contact. The region may be encircled on the image using a digital pen, or marks can be made on the image by the physician to delineate the region of interest. The number of points of tissue contact, or the distance between the points is specified by the physician. The system 20 then drives the catheter tip to points within the region. There are many possible means by which the system can sense contact with tissue. A larger amplitude ECG signal can indicate surface contact. The response of the catheter to steering and advancing commands, as determined through image analysis and/or localization data, will change when the catheter contacts tissue. For example, a very flexible region proximal to the catheter tip will buckle if the catheter is advanced with the tip constrained against tissue. The buckling can be sensed by analysis of the catheter image, or by comparing the output of localization sensors located in the catheter tip and in the section proximal to the tip. Another indication of tip contact with tissue is the change in electrical impedance between the catheter tip and a ground pad located on the patient's skin. The physician can verify contact with a computer click. Once contact is made, data is collected or therapy is given. Data may be the ECG signal and localization information. Therapy may be drug delivery or tissue biopsy.

Ablation of tissue to eliminate a cardiac arrhythmia is often the therapeutic goal of the electrophysiologist. The system can facilitate automated mapping to locate the focus of the arrhythmia. The ability of the System to automatically guide the catheter back to a point for ablation or recheck of the ECG signal before or after ablation is of key importance for the present invention. The computer stores the three dimensional location and ECG signal at all points, and can recall this data and return to locations of interest to within about 1 mm accuracy. In many cases, lines of ablation or even circles of ablation are required. The system 20 can greatly facilitate formation of these continuous lesions. For example, in the ablation of atrial flutter, a line of block is required from the annulus of the tricuspid valve to the inferior vena cava. The system 20 steers the catheter into contact with tissue, while the advancer retracts the catheter to form the line. Ablation can be continuous during catheter retraction, or the system 20 can stop retraction and ablate at a series of closely spaced points. After the line is created, the system 20 can re-map the area to confirm conduction block. Circles of block outside the ostium of the pulmonary veins are required to ablate focal atrial fibrillation. These circles can be specified by the physician and the system 20 can then drive the tip of the catheter continuously or to points along the circular paths. Some chronic atrial fibrillation requires lines connecting the circles and other complex lines of block, which are greatly facilitated by the system 20.

Catheter tip steering by the system 20 enables a very flexible catheter segment proximal to the tip. This flexibility allows the catheter to be doubled back on itself, which is an important requirement in some anatomical settings. For example, when a catheter is introduced into the left atrium via a trans-septal puncture, it must be able to double back to ablation sites on the left side of the septum. When the left atrium is addressed from the arterial system, the catheter must go up through the aorta, down through the left ventricle, then up through the mitral valve into the left atrium. Once in the atrium, it must be able to address any specified points. In the prior art, there is no manually operated catheter which can address all tissue points in the left atrium required to ablate and cure atrial fibrillation. The System allows tissue contact to be easily made at any point within the left atrium, facilitating a non-surgical cure for atrial fibrillation.

The navigation of a magnetically steered catheter with a localization device mounted at its tip can be automated by using a feedback control system that is based on the interaction between several forces. In general, the catheter assumes an equilibrium configuration determined by a balance between elastic, magnetic, gravitational and constraint forces (the latter may include frictional forces). Navigation using localization data has the desirable advantage (to both physician and patient) of minimizing radiation exposure to X-ray imaging.

The general equations governing deformations and equilibria of catheters are known, see for example W. Lawton, R. Raghavan, S. R. Ranjan and R. R. Viswanathan, ‘Ribbons and groups: a thin rod theory for catheters and filaments’, Journal of Physics A, Vol. 32, No. 9, p. 1709-1735, 1999, incorporated herein by reference, and are summarized below. These equations may be exploited to construct a feedback control system for catheter navigation. Several cases may be distinguished and are explained below.

A thin rod such as a catheter undergoes deformations that predominantly involve only local bend and twist, with negligible stretching effects. These deformations may be characterized by studying the change of local frame along the catheter. Specifically, let the rod be parameterized by arc length s along its curve, such that u(s) is the local (unit) tangent vector along the curve in its equilibrium or reference configuration. The local cross section (specified by a vector v(s) lying in the plane of the cross section) together with local tangent vector defines a local frame everywhere along the length of the rod.

When the rod deforms, the local frame is rotated in space. The (local) rotation may be described by a 3×3 rotation matrix M(s). The strain associated with this deformation (the derivative of M(s) pulled back to the undeformed or reference curve) is characterized by a 3×3 anti-symmetric rotation rate matrix Ω(s):
Ω(s)=M^{T}(s) dM(s)/ds (1)
where the superscript “T” denotes a matrix transpose. The matrix Ω(s) has 3 independent non-zero elements that form a vector ω(s) with components (ωₓ, ω_{y}, ω_{z}):

$$Ω = ( \begin{matrix}0 & - {ω}_{z} & {ω}_{y} \\ {ω}_{z} & 0 & - {ω}_{x} \\ - {ω}_{y} & {ω}_{x} & 0\end{matrix} ) \qquad ( 2 )$$

The local elastic energy density associated with the thin rod deformation may be written in terms of the twist and bend components of the strain, respectively ωₜ=(ω.u)u and ω_{b}=ω−(ω.u)u, in a manner that is familiar from the literature:

$$J ( s ) = \frac{1}{2} {G}_{s} I {\vert {ω}_{t} \vert}^{2} + \frac{1}{2} {E}_{y} I {\vert {ω}_{b} \vert}^{2} \qquad ( 3 )$$

where I is the bending moment of area of the cross section, Gₛ is twice the shear modulus and E_{Y} is the Young's modulus of the material of the rod. The energy density may be rewritten in the form

$$J ( s ) = \frac{1}{2} {ω}^{T} ( s ) Q ( s ) ω ( s ) \qquad ( 4 )$$

where ω is written as a column vector and Q(s) is a 3×3 matrix that characterizes the material properties and geometry of the rod. In the presence of applied forces and torques, the equations of equilibrium of the rod follow from minimizing equation (4) subject to the applied loading as a constrained optimization problem. The resulting equilibrium equations may be derived from standard constrained optimization methods and may be shown to be:
dr(s)/ds=M(s)u(s)
dM(s)/ds=M(s)Ω(s)
d(Q(s)ω(s))/ds=(M^{T}λ)×u−ω×Qω+M^{T}dτ/ds (5)
where the symbol “x” denotes vector cross product, r(s) is the position vector along the deformed rod, dλ/ds is the applied force density and dτ/ds is the applied torque density along the length of the rod.
These nonlinear equations of equilibrium must be supplemented by suitable boundary conditions. In general we have a nonlinear two-point boundary value problem to solve. For example, consider a rod of total (arc) length L. When the rod is fixed in position at one end r(s=0)=r₀ and a known force f(L) and torque τ(L) are applied at the other end (s=L), the rotation rate and position vectors satisfy the boundary conditions
r(s=0)=r₀
Q(0)ω(0)=M^{T}(0)(r(L)−r(0))×f(L)+M^{T}(L)τ(L)
M(L)Q(L)ω(L)=τ(L) (6)
Equations (5) in this case must be integrated with the boundary conditions (6) to arrive at a self-consistent solution.
As a second example, if one end of the rod (s=0, say) is constrained in both position and orientation, and the other end (s=L) is constrained in position, we have
r(s=0)=r₀
M(s=0)=M₀
r(s=L)=r_{L} (7)
In this case the boundary condition for ω(0) is
ω(0)=0 (8)

(since the orientation is constrained at this end, it cannot change during loading and the rotation rate must be zero at this end). The solution for the force and torque applied at the other end must be chosen so as to be consistent with equations (7) and (8) when equations (5) are integrated.

In the case of a “contact-free” tip, the localization device provides position x as a vector in three dimensional space and an orientation that may be written as 3×3 matrix M, both at the catheter tip. When there is no tip contact so that the tip is free, the weight of the seed or tip magnet provides a vertically downward force f=mg minus the weight of displaced blood, where m is the mass of the seed magnet. At the same time, the presence of a magnetic field provides a torque τ=μ×B, where μ is the dipole moment of the seed magnet and B is the external magnetic field in the tip region (assumed homogeneous in this region for the present discussion, although this assumption may be relaxed and dealt with as known to those familiar with the art). Thus the force and torque at the tip are known.

The catheter's last point of contact with the cardiac chamber/vessel wall may be assumed to be in the region of the last branch point of vessel/chamber branching. From pre-operative angiograms registered to localization system coordinates, a plane transverse to the parent vessel may be constructed at the branch point. The last point of contact of the catheter may be taken to be somewhere within this plane P.

Using the known catheter tip position and orientation and force and torque, as well as elastic constants for the catheter (assumed known from earlier measurements of dimensions and material properties), the thin rod equations of thin rod equations (5) above may be integrated back until the catheter intersects the plane P. This yields a position x₀ and orientation M₀ at the last point of contact, as well as catheter length to last point of contact. The entire configuration and length of the catheter between the locations x₀ and x is now known.

The navigation problem may be formulated as follows: given the present location x and external magnetic field B at the tip, find a new field B₂ and a catheter advancement δs that will steer the catheter to a new desired location x₂. This is an incremental problem, so that δB=(B₂−B) and δx=(x₂−x) are small quantities. Larger changes in target or tip position x may be divided into smaller increments so that we always have an incremental problem at every step of the process.

The incremental problem of finding a combination of δB and δs which results in the desired positional change δx may be solved by the method of linear response. In this method, the thin rod equations (5) are integrated from point of last contact to the tip over the length of the catheter between these points. Thus, points along the length of the catheter are parametrized by a variable s; for convenience this may be taken to be a length variable. Catheter deformations have negligible stretch and so a parametrization based on length is a convenient choice. Let s₀ and s₁ be the length parameters of the last point of contact and the tip, respectively. We perform the following steps: (a) Using the same force and torque values, we assume a value for the quantity δs₁ and integrate the thin rod equations from s₀ to (s₁+δs₁), which will give us a new tip position y which differs from x by an amount δy=(y−x); (b) likewise, assuming a change δC in magnetic field one component at a time and integrating the thin rod equations from s₀ to s₁ gives new positional changes δz at the tip. These results may be summarized conveniently in matrix form as
δy=L[δs δB^{T}]^{T} (9)
where the boldface vectors are taken to be column vectors and the superscript “T” denotes a matrix transpose. The matrix L here is called the linear response matrix and its entries are obtained directly as coefficients dictating proportionality between changes in δs and δB, and those in tip position δz, as obtained from the results of steps (a) and (b) above. In general L is a 3×4 matrix and it describes the resulting change in tip position upon making changes in the catheter length parameter and the applied magnetic field. In practice, changes in the field B usually arise from changes in direction of the field alone. In other typical cases, it is desirable that the change in field is confined to a plane containing the present tangent vector to the catheter at its tip and the desired incremental target point. In both of these cases, the change in field δB effectively has only two degrees of freedom and may be written in the form
δB^{T}=E δBₜ^{T} (10)
where δBₜ has only 2 components and E is a known or given 3×2 matrix. Equation (2) may be used to write equation (1) in the form
δy=R[δs δBₜ^{T}]^{T} (11)
where R is now a 3×3 matrix.

Given a desired change in position δx at the tip, equation (3) may be inverted to find the corresponding set of changes δs and δB in inserted length of catheter and applied magnetic field respectively which yield the desired change in tip position. This solves the incremental control problem.

In a second situation, the force at the catheter tip is known. For convenience, it may sometimes be desirable to incorporate a miniature force sensing device at the catheter tip which measures contact force at the tip, if any. In this case, in addition to localization information, we know the total or net force and torque acting on the catheter tip (the net force acting on the tip is the vector sum of the contact force and the gravitational force).

Then the control problem is solved incrementally in exactly analogous manner to that described above with respect to “contact-free” tip

In a third situation the contact force at the tip is unknown. If there is no force sensor at the catheter tip, and the catheter is making contact with tissue at the tip, the tip force is unknown. In this case, we can only approximately solve the control problem due to insufficient data.

Device localization gives us position and orientation x and M at the tip, and the known magnetic field gives the torque acting at the tip. Registration with a pre-operative image or image taken at the start of the procedure would tell us if tissue contact is being made at the tip location.

Assuming that the last point of contact occurs at the last vessel branching, position x₀ and orientation M₀ at the last contact point may be assumed from vessel geometry, or may be obtained to a good approximation from X-ray fluoroscopy images taken at occasional intervals during the procedure. The control algorithm proceeds as follows: (i) determine the configuration and length of the catheter between x₀ and x (this process also involves determining the tip force by mathematical means); (ii) using the configuration information so obtained, the procedure detailed in above with respect to the “contact-free” tip solves the navigation control problem.

Step (i) is implemented as follows. First, we note that the torque (due to the magnetic field) at the catheter tip is known (since orientation there is known). Assuming a value l for the length of catheter between x₀ and x, the thin rod equations (5) are integrated over this length using zero applied force and the known torque at the tip. This will yield a position xₜ for the catheter tip. Next the difference (x−xₜ) is discretized into a given number of small increments δx. Corresponding to these incremental end-point displacements, a step-wise procedure of determining configurations incrementally is followed. By applying unit or known force increments component-wise, a linear response matrix P giving the linear relation between end-point displacement δx and incremental end-point force δf is obtained:
δx=P δf
so that δf=P⁻¹ δx is the incremental force needed to produce the incremental end-point displacement δx at each step. The thin rod equations when integrated with this applied incremental force at each step gives the equilibrium configuration at each step, until finally the catheter end-point or tip has moved to the location x under the influence of a net force f and known torque. Correspondingly the catheter tip orientation will be given by an orientation matrix Mₑ which will in general be different from the known tip orientation M. Ideally, a consistent solution requires that M and Mₑ match exactly. Therefore we form a quantitative measure of the computed and known tip orientations:define
q=[Tr(M)−Tr(Mₑ)]²+w[∥M−M^{T}∥²+∥Mₑ−Mₑ^{T}∥²]
where ∥ ∥ denotes the matrix norm (square root of sum of squares of matrix entries).

We repeat the above process over a range of assumed values (suitably discretized) for l (a good starting guess for l is the distance l₀=|x−x₀|), with the range l₀ to 4l₀/3 being a preferred range of values. The catheter configuration corresponding to the length value with the minimum deviation q* between computed and known tip orientations is taken to be the correct catheter configuration.

It is important to note that this estimate may be further refined if desired by starting from a variety of possible choices for last point of contact data (x₀, M₀), finding the configuration with the least q value (q*) each time, and finally from this set of configurations selecting that with the least value of q*.

Now step (i) is complete, and as mentioned earlier step (ii) may be implemented above with respect to a “contact-free” tip.

As shown in FIG. 9, which is a schematic representation of the device geometry, a device such as a catheter 301 is inserted into a patient's blood vessels 307. The plane 311 of last vessel branching before the tip of the catheter 317 (with coordinate x) is reached is determined from fluoroscopic imaging, either pre-operatively or intra-operatively. The point of last contact xo is determined by finding an equilibrium configuration for the device which intersects plane 311.

As shown in FIG. 10, a process flow chart for the feedback controlled navigation system, localization data (at the device tip), contact force data if any and desired target point information are input into the system in step 610, as is information about a branch plane at the point of last contact/vessel branching in step 613. The current equilibrium configuration of the catheter between the point of last contact and the tip is then determined computationally in step 616. The vector difference between current tip location and desired target location is divided into a specified number of increments in step 619. The linear response matrix for the current configuration is found and incremental magnetic field changes and device advancement values which would result in the desired incremental change in tip location are applied in step 622. The incremental process is continued in step 625 and the entire process is repeated until all increments have been applied. In another embodiment of the invention, the entire incremental process may be repeated up to a specified number of iterative improvements or until the desired tip location is reached to within a specified tolerance. In yet another embodiment of the invention, at each increment, the step size may be adaptively determined depending upon the deviation between desired and achieved tip locations at certain intermediate steps in the process.

1. An automated system for navigating a medical device through the lumens and cavities in an operating region in a patient, the system comprising:
an elongate medical device, having a proximal end and a distal end adapted to be introduced into the operating region;
an imaging system for displaying an image of the operating region, including a representation of the distal end of the medical device in the operating region;
a localization system for determining the position of the medical device in a frame of reference translatable to the displayed image of the imaging system;
a magnetic navigation system for orienting the medical device in a selected direction in the operating region through the interaction of magnetic fields associated with the medical device inside the operating region and at least one external source magnet outside the patient's body;
an advancer acting on the proximal end of the medical device for selectively advancing the distal end of the medical device;
an input device for receiving at least a destination for the distal end of the device input by the user using the displayed image of the imaging system;
and a controller, responsive to the destination input by the user, for operating the magnetic navigation system to orient the distal end of the medical device in the proper orientation to reach the selected destination, and once the distal end of the medical device is in the proper orientation, operating the advancer to advance the distal end of the medical device to the destination input by the user.

2. The system of claim 1 wherein the controller only operates the advancer after the magnetic navigation system has oriented the medical device in the selected direction.

3. The system of claim 1 wherein the imaging system includes an x-ray source and an x-ray detector for imaging the operating region.

4. The system of claim 1 wherein the imaging system is an MRI system that applies a magnetic field to the operating region, and wherein the magnetic navigation system comprises at least one coil in the distal end of the device for selectively changing the magnetic moment of the medical device.

5. An interventional medical system for guidance of medical devices within patient body lumens and cavities comprising: a digitally controlled medical device advancer; a digitally controlled medical device tip steering mechanism; a means for determining the spatial coordinates of at least one point on the medical device adjacent the distal tip; an imaging system which displays images of the operating region which include an image of the catheter superimposed on tissue; a user interface that allows the operator to interact with the images to specify at least one of a location or a path; a control computer that interprets operator specifications, and operates the medical device advancer and the medical device tip steering mechanism to guide the medical device to the specified location or along the specified path, and update the images of the catheter and tissue.

6. The system of claim 5 in which the catheter advancer includes a digitally controlled motor.

7. The system of claim 5 in which two or more catheter advancers simultaneously control two or more catheters.

8. The system of claim 5 in which the catheter steering mechanism is magnetic coupling between digitally controlled magnets external to the patient and a fixed magnetic material in the catheter tip.

9. The system of claim 5 in which the catheter steering mechanism is magnetic coupling between a fixed magnet external to the patient and a digitally controlled magnetic moment in the catheter tip.

10. The system of claim 5 in which the catheter steering mechanism is actuation of electrostrictive or magnetostrictive elements in the catheter tip.

11. The system of claim 5 in which the catheter steering mechanism is a combination of magnetic coupling between a fixed magnet external to the patient and a digitally controlled magnetic moment in the catheter tip, and actuation of electrostrictive elements in the catheter tip

12. The system of claim 5 in which the determination of spatial coordinates of the catheter is made by computer processing of digital images of the operating region.

13. The system of claim 5 in which the determination of spatial coordinates of the catheter is made by computer analysis of signals transmitted or received from within the catheter.

14. The system of claim 5 in which the images are X-ray fluoroscopic digitized or digital images.

15. The system of claim 5 in which the images are MRI images updated faster than once per second.

16. The system of claim 5 in which the images are local endoscopic images.

17. The system of claim 5 in which the catheter is steered and advanced or retracted to automatically maintain the relative position of the catheter tip and a point on tissue which is moving within the patient's body.

18. The system of claim 5 in which the images are local ultrasound images.

19. The system of claim 5 in which the images are from a pre-operative MR or CT scan.

20. The system of claim 5 in which the images are a combination of real time and pre-operative images.

21. A method for navigating a medical device through the body lumens and cavities of a patient, consisting of: observing the operating region and medical device on a composite medical image; specifying desired points or paths for the medical device on a user interface; navigation of a computer controlled steering and advancer means which advances the medical device to the specified points.

22. The method of claim 21 in which the catheter is retracted so that the tip moves along a specified line of tissue within the patient.

23. The method of claim 22 in which the a linear lesion is created to block atrial flutter.

24. The method of claim 21 in which the path is circular.

25. The method of claim 24 in which a circular of ablation is created adjacent the ostium of a pulmonary vein to block atrial fibrillation.

26. The method of claim 21 in which the catheter tip is moved in a path which keeps the tip stationary relative to a point on tissue which is moving within the patient.

27. The method of claim 26 in which the moving tissue is the heart.

28. The method of claim 27 in which the point on the heart is an ostium of a coronary blood vessel.

29. A method of navigating the distal end of a medical device in an operating region in a patient, the method comprising:
displaying an image of the operating region;
localizing the position of the distal end of the medical device in the operating region;
superposing a representation of the distal end of the medical device on the displayed image of the operating region;
accepting a destination for the distal end of the medical device input by the user using the displayed image of the operating region;
and iteratively localizing the position of the distal end of the medical device, orient determining the orientation for the distal end of the medical device to reach the destination input by the user, and orienting the distal end of the medical device in the determined orientation; and automatically advancing the distal end of the medical device toward the destination until the distal end of the medical device is at the input destination.

30. A method of navigating the distal end of a medical device in an operating region in a patient, the method comprising:
displaying an image of the operating region;
localizing the position of the distal end of the medical device in the operating region;
superposing a representation of the distal end of the medical device on the displayed image of the operating region;
accepting a destination for the distal end of the medical device input by the user using the displayed image of the operating region;
determining the orientation for the distal end of the medical device to reach the destination input by the user, and orienting the distal end of the medical device in the determined orientation;
automatically advancing the distal end of the medical device toward the destination, and iteratively localizing the position of the distal end of the medical device, orient determining the orientation for the distal end of the medical device to reach the destination input by the user, and orienting the distal end of the medical device in the determined orientation; and automatically advancing the distal end of the medical device toward the destination until the distal end of the medical device is at the input destination.

31. A method of navigating the distal end of a medical device in an operating region in a patient, the method comprising:
displaying an image of the operating region; localizing the position of the distal end of the medical device in the operating region;
superposing a representation of the distal end of the medical device on the displayed image of the operating region;
accepting a destination for the distal end of the medical device input by the user using the displayed image of the operating region; and iteratively localizing the position of the distal end of the medical device, determining the orientation for the distal end of the medical device to reach the destination input by the user, and advancing the catheter upon input from the user.

32. The method of claim 30 wherein the speed that the user can advance the medical device depends upon how close the orientation of the medical device is to the determined orientation.

33. A method of navigating the distal end of a medical device in an operating region in a patient, the method comprising:
displaying an image of the operating region;
localizing the position of the distal end of the medical device in the operating region;
superposing a representation of the distal end of the medical device on the displayed image of the operating region;
accepting a path for the distal end of the medical device input by the user using the displayed image of the operating region;
and iteratively localizing the position of the distal end of the medical device, determining the orientation for the distal end of the medical device to follow the path input by the user, and orienting the distal end of the medical device in the determined orientation; and
automatically advancing the distal end of the medical device along the path.

34. The method of claim 33 wherein the path is input as a series of points.

35. The method according to claim 33 further comprising the step of making a preoperative imaging showing the patients' vasculature; and wherein the step of orienting and iteratively localizing the position of the distal end of the medical device, determining the orientation for the distal end of the medical device to reach the destination input by the user and orienting the distal end of the medical device in the determined orientation includes orienting the distal end of the device to travel in the patient's vasculature in the preoperative image.

36. A method of navigating the distal end of a medical device in an operating region in a patient, the method comprising:
creating a preoperative image of the operating region;
localizing the position of the distal end of the medical device in the operating region;
superposing a representation of the distal end of the medical device on the displayed image of the operating region;
accepting a destination for the distal end of the medical device input by the user using the displayed image of the operating region;
and iteratively localizing the position of the distal end of the medical device, orient determining the orientation for the distal end of the medical device to reach the destination input by the user, and orienting the distal end of the medical device in the determined orientation; and automatically advancing the distal end of the medical device toward the destination until the distal end of the medical device is at the input destination.

37. A method of navigating the distal end of a medical device in an operating region in a patient, the method comprising:
creating a preoperative image of the operating region;
localizing the position of the distal end of the medical device in the operating region;
superposing a representation of the distal end of the medical device on the displayed image of the operating region;
accepting a destination for the distal end of the medical device input by the user using the displayed image of the operating region;
and iteratively localizing the position of the distal end of the medical device, orient determining the orientation for the distal end of the medical device to reach the destination input by the user, and orienting the distal end of the medical device in the determined orientation; and automatically advancing the distal end of the medical device toward the destination until the distal end of the medical device is at the input destination.

38. A method of controlling movement and navigation of a medical device inserted into a patient, said method comprising: (a) localization of the device tip, said device exhibiting deflection capability in response to application of external magnetic fields, (b) using localization information together with device elasticity properties to magnetically guide the device tip to a desired location by suitable application of magnetic fields together with device advancement/retraction, and (c) said guidance implemented by the use of feedback control algorithms based on the construction of mathematical models of the interaction of device elasticity and applied external forces and torques on said device.

39. The method of claim 38, where said deflection capability is achieved by the use of a permanent magnet located within said device.

40. The method of claim 38, where said external forces in said mathematical models include gravitational forces.

41. The method of claim 38, where said external forces and torques included in said mathematical models arise from application of external magnetic fields.

42. The method of claim 38, where said feedback control algorithms utilize information obtained from pre-operative X-ray imaging.

43. The method of claim 38, where said feedback control algorithms utilize information obtained from intra-operative X-ray imaging.

44. The method of claim 38, where said device includes at least one force sensor at the device tip providing contact force data for incorporation into said mathematical models.

45. The method of claim 44, where said mathematical model includes tip contact forces.

46. The method of claim 45, where said mathematical model includes tip contact forces.

47. The method of claim 46, where said feedback control algorithms utilize contact force data.

48. The method of claim 47, where said feedback control algorithms utilize contact force data.

49. The method of claim 38, where said device advancement/retraction is achieved by computer control of an advancement/retraction system.

50. The method of claim 38, where said medical device is a catheter.

51. A system for closed-loop feedback control of medical device steering and advancement, said system including: (a) a device localization sub-system for obtaining localization information at the device tip, (b) computer control of device advancement/retraction, (c) computer-controlled means of magnetic field application, (d) software and hardware for feedback control of catheter motion, said software including feedback control algorithms based on physics-based mathematical models of device elasticity, (e) automated computer control of device advancement and magnetic field application achieved by the use of localization data in conjunction with feedback control algorithms, and (f) a manual over-ride option for use in manual or semi-automated mode.

52. The system of claim 51, where said feedback control algorithms utilize information obtained from pre-operative X-ray imaging.

53. The system of claim 51, where said feedback control algorithms utilize information obtained from intra-operative X-ray imaging.

54. The system of claim 51 which includes a device localization subsystem for obtaining localization and orientation information at the device tip.
